(19) United States
(12) Patent Application Publication
(10) Pub. No.: US 2024/0066232 A1
(43) Pub. Date: Feb. 29, 2024

(54) DOSING SYRINGE AND METHODS OF USING SAME

(71) Applicant: Zachary O Knable, Medford Lakes, NJ (US)

(72) Inventor: Zachary O Knable, Medford Lakes, NJ (US)

(21) Appl. No.: 17/822,159

(22) Filed: Aug. 25, 2022

(51) Int. Cl. A61M 5/315

(52) U.S. Cl. CPC A61M 5/31536; A61M 5/31501; A61M 5/31511; A61M 5/31563; A61M 2005/31508

(57) ABSTRACT

A dosing syringe includes a barrel extending longitudinally between a proximal end and a distal end, the barrel having a plurality of sections represented by colored indicators, each of the plurality of sections corresponding to a medically-relevant dose, a plunger translatable within the barrel, the plunger including a plurality of segments represented by colored markings that correspond to the colored indicators of the plurality of sections, the plurality of segments further including a plurality of notches, each of the plurality of notches corresponding to one of the plurality of segments, and a tab mateable with one of the plurality of notches and configured to limit travel of the plunger within the barrel.

### FIELD OF THE INVENTION

The present disclosure relates generally to syringes. More specifically, the present disclosure relates to syringes and related methods that allow for easier dosing.

### BACKGROUND OF THE INVENTION

Several devices are known for the application of injectable medicaments up to the present time, most of them require the decanting of the content of the ampoules that contain them into the syringes to be injected directly into the patient or into the interior of systems of intravenous injection. The amount of medication to be administered may depend, at least in part, on the weights of the patient and the type of medication. This is particularly true in pediatric and neonatal applications where volume sensitivity is key.

Modern devices and techniques do not provide a quick and simple method of identifying and confirming that a certain amount of medication is being administered to the patient. Instead, a healthcare provider may need to load the syringe with medication and carefully monitor that the correct volume is being administered by observing the markings on the syringe. Such techniques take longer and are prone to user error.

### SUMMARY OF THE INVENTION

In some examples, a dosing syringe includes a barrel extending longitudinally between a proximal end and a distal end, the barrel having a plurality of sections represented by colored indicators, each of the plurality of sections corresponding to a medically-relevant dose, a plunger translatable within the barrel, the plunger including a plurality of segments represented by colored markings that correspond to the colored indicators of the plurality of sections, the plurality of segments further including a plurality of notches, each of the plurality of notches corresponding to one of the plurality of segments, and a tab mateable with one of the plurality of notches and configured to limit travel of the plunger within the barrel.

In some examples, a method of dosing includes providing a barrel extending longitudinally between a proximal end and a distal end, the barrel having a plurality of sections represented by colored indicators, each of the plurality of sections corresponding to a medically-relevant dose, and a plunger translatable within the barrel, the plunger including a plurality of segments represented by colored markings that correspond to the colored indicators of the plurality of sections, the plurality of segments further including a plurality of notches, each of the plurality of notches corresponding to one of the plurality of segments, visually identifying a relevant dose using the colored indicators, and mating a tab to one of the plurality of notches.

### BRIEF DESCRIPTION OF THE DRAWINGS

Various embodiments of the presently described dosing syringes are disclosed herein with reference to the drawings, wherein:

FIG. 1 is a schematic illustration of a perspective view of a dosing syringe;

FIG. 2 is a top view of the dosing syringe of FIG. 1;

FIG. 3 is a side view of the dosing syringe of FIG. 1;

FIG. 4 is an end view of a tab of the dosing syringe of FIG. 1;

FIG. 5 is a schematic illustration of the color-coding or shading of the barrel and plunger; and

FIG. 6 is a schematic illustration of a perspective view of an alternative dosing syringe.

Various embodiments will now be described with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to syringes, conventional methods suffer from some shortcomings as discussed above.

There, therefore, is a need for further improvements to the devices and methods used to help facilitate proper and safer delivery of medication. Among other advantages, the present disclosure may address one or more of these needs.

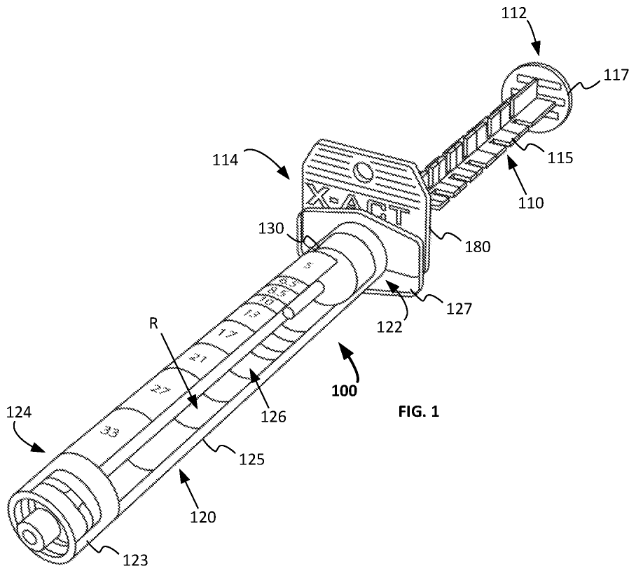

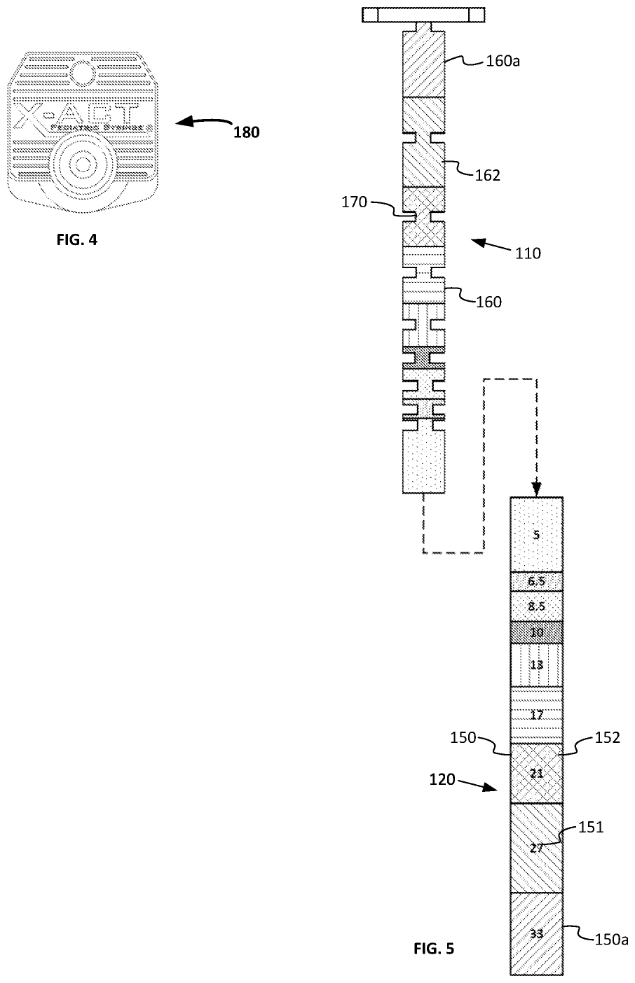

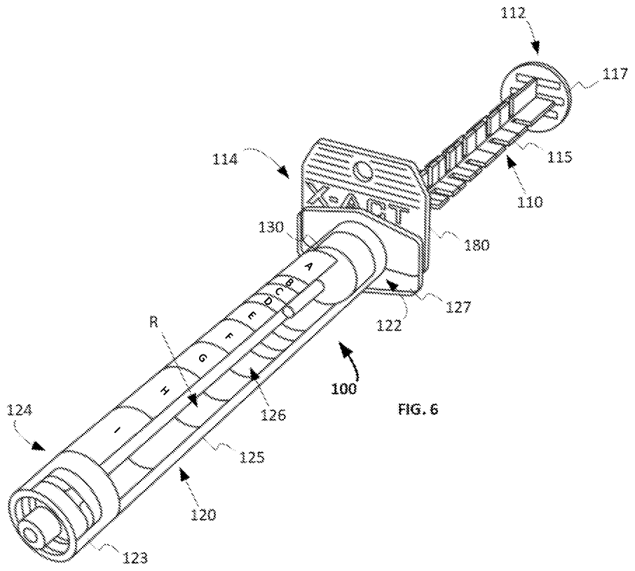

Reference is now made to FIGS. 1-4, which show a dosing syringe 100. Dosing syringe 100 generally comprises two main portions, a plunger rod 110 and a barrel 120. The end of barrel 120 may include a luer lock 123, luer slip (not shown), or a stacked needle (not shown). Plunger rod 110 generally extends between a proximal end 112 and a distal end 114, and comprises an elongated piston 115 extending between a thumbpress 117 and a coupler (not shown). In one embodiment, piston 115 has a cruciform cross-sectional shape. In one embodiment, thumb press 117 has a circular shape.

A cylindrical barrel 120 extends between proximal end 122 and distal end 124 and comprises a body 125 defining a lumen 126 for accepting a portion of plunger rod 110. As used herein, a “proximal” end means the end toward an operator and a “distal” end means the end away from the operator. Body 125 further comprises a flange 127 adjacent proximal end 122. Body 125 defines a reservoir “R” that holds a medicament, drug, saline, or other substance. In some examples, an internally threaded stopper 130 is disposed inside lumen 126 of body 125. In one embodiment, stopper 130 is made of an elastomeric material such as natural rubber, synthetic rubber, thermoplastic elastomers, or combinations thereof, and includes an opening to receive and mate with a coupler of plunger rod 110 by advancing the plunger rod inside the barrel lumen 126 and rotating at least one of coupler and stopper 130 relative to the other.

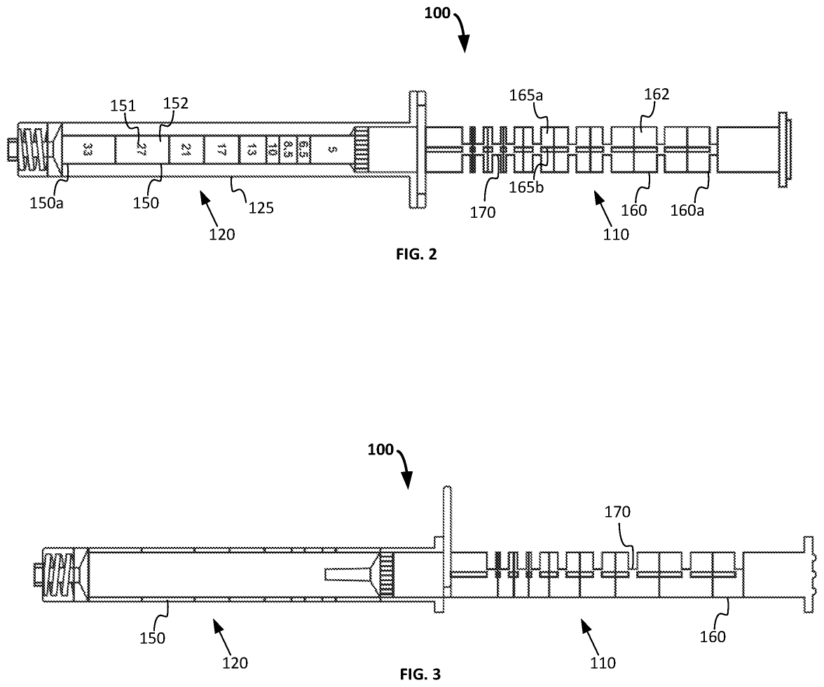

Turning to FIGS. 2-3, it will be understood that barrel 120 may include markings on its surface that divide the barrel into different sections 150. Each section 150 may include a dosing notation 151 (e.g., a notation corresponding with a specified volumetric dose) and/or a colored indicator 152. In some examples, each section 150 includes a unique dosing notation and a unique color or shading or indicia that matches the dosing notation. Each of the plurality of sections 150 may correspond to a medically-relevant dose. In the example shown, nine sections 150 correspond to nine weight-based doses: 5 kg, 6.5 kg, 8.5 kg, 10 kg, 13 kg, 17 kg, 21 kg, 27 kg, and 33 kg. In some examples, the 5 kg section is gray, the 6.5 kg section is pink, the 8.5 kg section is red, the 10 kg section is purple, the 13 kg section is yellow, the 17 kg section is white, the 21 kg section is blue, the 27 kg section is orange and the 33 kg section is green. The specific colors chosen for each of the sections may be altered as desired. For clarity in the black-and-white line drawings, FIGS. 1-3 illustrate the syringe barrel 120 and plunger 110 without colors. To more clearly illustrate the utility of the disclosure, FIG. 5 is presented where appropriate shading has been used to replace colors. Specifically, instead of color, the nine sections 150 of dosing syringe 100 are illustrated with nine unique patterns or shadings in FIG. 5. It will be understood that the actual sections 150 of dosing syringe 100 may include colors, patterns, shadings, markings, annotations, text, symbols or any combination thereof. Additionally, in some examples, the sections 150 correspond to segments of Broselow™ Tape/Broselow™ pediatric emergency tape or color-coded weight-based tapes.

It will be noted that sections 150 represent uneven increments (e.g., they are not, for example, divided into consistent volumetric increments, such as traditional 1 mL increments). The sections 150 may communicate the cumulative weight-based dosing volume that will be delivered to the patient. For example, the section labeled 5 kg communicates that the weight-based dose for 5 kg will be delivered to the patient. The section labeled 6.5 kg communicates that the volume from the 5 kg previous section will be delivered as well as the weight-based volume for the additional 1.5 kg from the adjacent section. For this reason, the “5 kg” section is shown larger than the “6.5 kg” section. In some examples, the dosing notation 151 and/or colored indicator 152 are disposed on opposing surfaces of the barrel 120 so that a user can quickly identify the sections without needing to flip the syringe. In at least some examples, the colored indicator 152 is circumferentially disposed so that the barrel 120 is divided into colored rings.

Plunger rod 110 and specifically piston 115 may likewise be divided into a number of segments 160 having colored markings 162 that correspond to the colored indicators 152 of the plurality of sections 150. In at least some examples, each colored marking 162 will correspond to a unique colored indicator 152. In at least some examples, the correspondence means that a particular section 150 and a particular segment 160 have the same color, which is unique from any other segment or section. In examples where the piston 115 has a cruciform shape, each of the first and second members 165a,165b may be partially or entirely covered with colored markings 162. As best shown in FIGS. 2 and 5, the distalmost section 150a of barrel 120 corresponds to the proximal-most segment 160a of plunger 110, and each successive section corresponds with a successive segment as moving inwardly to the middle of the device. Each of the plurality of segments 160 may further include one or more notches 170. These notches 170 may be defined in the first member and/or the second member of the piston. In some examples, notches 170 includes notches on both first and second members 165a,165b. In some examples, a plurality of notches 170 are formed at predetermined longitudinal extents, the plurality of notches including one or more notches on the first member 165a, and one or more notches on the second member 165b.

The notches 170 may be defined in these segments coincident with an appropriate amount of travel of the plunger within the barrel to achieve delivery of the cumulative volume noted on the barrel. For example, a notch in the green-colored segment will signal the travel of the piston so that a cumulative volume needed for the weight-based dose of a 33 kg patient is delivered. A notch within the orange-colored segment will signal the travel required so that a cumulative volume needed for the weight-based dose of a 27 kg patient is delivered.

As shown in FIGS. 1-4, a removable tab 180 may be coupleable with one of the notches to limit the travel of the piston. In some examples, tab 180 is a square-shaped, rectangular, hexagonal or polygonal member with a width that is suitable for mating within any of the notches. In at least some examples, tab 180 is larger than flange 127 or thumbpress 117. Tab 180 may be substantially planar and may have a flat surface that will abut flange 127 and limit further movement of the plunger when coupled thereto. In at least some examples, tab 180 may have an initial position that is distal to all segments 160 (as shown in FIG. 3) to prevent any travel of the plunger within the barrel until the operator removes the tab from the initial position and places it at a desired secondary position based on intended dosing.

In use, the operator may determine the volume of medication to be delivered to the patient based on medication strength, patient weight, patient condition and/or any other criteria. Once the volume is decided, the user may look at the barrel to visually identify or locate the intended volume (e.g., by looking for the 17 kg notations) and identify the appropriate color on the barrel. For example, if the operator decides that the 17 kg dose is required, then they will see that the intended volume is coded with a white section 150 on the barrel. The user may then use removable tab 180 and place it, or couple it to, in the corresponding white segment 160 of the plunger. In some examples, the tab 180 will be coupled to one or more members of the plunger (e.g., to the first and second members of a cruciform plunger). Once properly mated with the notch on the plunger, there is no need to further hold or manipulate the tab. When delivering the medication, the user may press on the thumbpress to actuate the plunger into the barrel. The plunger (along with the attached tab) will travel through the barrel until the tab 180 abuts flange 127 of the barrel, at which point the 17 kg weight-based dose will be completely delivered either directly into the patient or into an intravenous catheter. The tab 180 will impede the user from delivering any additional medication by limiting any further movement of the plunger. By using the systems and techniques described herein, the user may use the same dosing syringe to deliver any of a number of predetermined volumes, and may reduce the risk of inaccurate dosing.

It is to be understood that the embodiments described herein are merely illustrative of the principles and applications of the present disclosure. For example, the syringe has been described as being color-coded, patterns, notations, icons or other communicative elements may be used on the barrel, the plunger or both. Additionally, the tab may take other shapes or forms, and may slide on, mate with, or translate on the plunger in other ways. A dosing syringe may also be adapted to any other incremental dosing system, such as the Handtevy Pediatric Resuscitation System or other non-uniformed dosing systems, by adjusting the spacing of the sections. A more generic notation system is shown in FIG. 5 and it will be understood that, the lengths of the sections that correspond to “A”, “B”, “B”, “C” etc. may be adjusted as needed to coincide with a desired dosing system and/or range.

Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A dosing syringe, comprising:
a barrel extending longitudinally between a proximal end and a distal end, the barrel having a plurality of sections represented by colored indicators, each of the plurality of sections corresponding to a medically-relevant dose;
a plunger translatable within the barrel, the plunger including a plurality of segments represented by colored markings that correspond to the colored indicators of the plurality of sections, the plurality of segments further including a plurality of notches, each of the plurality of notches corresponding to one of the plurality of segments; and
a tab mateable with one of the plurality of notches and configured to limit travel of the plunger within the barrel.

2. The dosing syringe according to claim 1, wherein the plurality of sections includes unequal sections.

3. The dosing syringe according to claim 1, wherein the plurality of sections includes sections of non-uniformly-distributed volumes.

4. The dosing syringe according to claim 1, wherein the plurality of sections represents nine different volumes.

5. The dosing syringe according to claim 1, wherein the plurality of sections includes weight-based dosing sections corresponding to 5 kg, 6.5 kg, 8.5 kg, 10 kg, 13 kg, 17 kg, 21 kg, 27 kg, and 33 kg.

6. The dosing syringe of claim 1, wherein each of the colored indicators on the barrel corresponds to only one of the colored markings on the plunger.

7. The dosing syringe of claim 1, wherein the plunger includes a first member and a second member, the first member and the second member collectively having a cruciform cross-section, and wherein the plurality of notches includes notches on both the first member and the second member.

8. The dosing syringe of claim 1, wherein the plunger includes a first member and a second member, the first member and the second member collectively having a cruciform cross-section, and wherein the plurality of notches includes one notch on the first member and two notches on the second member.

9. The dosing syringe according to claim 1, wherein the plunger further includes a thumbpress that is provided at a proximal end of the plunger, said thumbpress being wider than a lumen of the barrel.

10. The dosing syringe of claim 9, wherein the tab is larger than the thumbpress.

11. The dosing syringe of claim 1, wherein the barrel includes a flange at the proximal end, and the tab includes at least one flat-surface configured to abut the flange.

12. The dosing syringe of claim 1, wherein the barrel includes a flange at the proximal end, and the tab is larger than the flange.

13. The dosing syringe of claim 1, wherein the tab is substantially flat.

14. The dosing syringe of claim 9, wherein each of the colored indicators are unique.

15. A method of dosing comprising:
providing a barrel extending longitudinally between a proximal end and a distal end, the barrel having a plurality of sections represented by colored indicators, each of the plurality of sections corresponding to a medically-relevant dose, and a plunger translatable within the barrel, the plunger including a plurality of segments represented by colored markings that correspond to the colored indicators of the plurality of sections, the plurality of segments further including a plurality of notches, each of the plurality of notches corresponding to one of the plurality of segments;
visually identifying a relevant dose using the colored indicators; and
mating a tab to one of the plurality of notches.

16. The method of claim 15, wherein the plurality of sections includes weight-based dosing sections corresponding to 5 kg, 6.5 kg, 8.5 kg, 10 kg, 13 kg, 17 kg, 21 kg, 27 kg, and 33 kg.

17. The method of claim 15, wherein visually identifying a relevant dose using the colored indicators includes identifying a unique colored indicator on the barrel.

18. The method of claim 15, wherein the plunger includes a first member and a second member, the first member and the second member collectively having a cruciform cross-section, and wherein mating a tab to one of the plurality of notches includes placing the tab through notches on both the first member and the second member.

19. The method of claim 15, wherein the barrel includes a flange at the proximal end, and further comprising the step of pushing a thumbpress until the tab abuts the flange.

20. The method of claim 15, wherein the barrel includes a flange at the proximal end, and further comprising the step of limiting travel of the plunger via the tab.
